(19) United States
(12) Patent Application Publication
(10) Pub. No.: US 2011/0187012 A1
(43) Pub. Date: Aug. 4, 2011

(54) AUTOMATIC MULTIFUNCTIONAL LIPOSOME MANUFACTURING DEVICE

(75) Inventors: Tetsuro Yoshimura, Mie (JP); Masatoshi Hashimoto, Mie (JP)

(73) Assignee: Hashimoto Electronic Industry Co., Ltd., Matsusaka-shi, Mie (JP)

(21) Appl. No.: 13/124,048

(22) PCT Filed: Oct. 13, 2009
(86) PCT No.: PCT/JP2009/067736

§ 371 Date: Apr. 13, 2011

(30) Foreign Application Priority Data
Oct. 14, 2008 (JP) 2008-265284

(51) Int. Cl. B01J 13/02; B01J 19/00; B06B 1/00; A61J 3/07; A61K 9/127

(52) U.S. Cl. 264/4.3; 422/198; 422/128; 264/4.6

(57) ABSTRACT

Provided is a device for formulating a large quantity of many kinds of liposomes quickly and efficiently using a small amount of organic solvent through computer-based automatic control. To this end, an automatic multifunctional liposome manufacturing device (1) is equipped with a cylindrical reaction vessel (2), an eccentric motor (3), a heater (4), a vacuum pump (10), a syringe pump (SP3) for supplying an organic solvent into a reaction space, a syringe pump (SP4) for supplying an aqueous solution into the reaction space, an ultrasonic processor (6), and a computer (15) for automatic control of the individual mechanisms in accordance with a program.

### TECHNICAL FIELD

The present invention relates to an automatic multifunctional liposome manufacturing device, which includes an eccentric motor, an ultrasonic processor, and a computer for automatic control of the operation thereof.

### BACKGROUND ART

Liposomes are closed vesicles composed of a lipid bilayer membrane. Liposomes resembling the biological membrane have been used as a variety of research materials to date.

Water-soluble active ingredients, antibodies, enzymes, genes and so on may be enclosed in the aqueous phase of liposomes. Also, oil-soluble proteins or active ingredients may be kept in the bilayer membrane of liposomes. Also DNAs or RNAs may be bound to the surface of the bilayer membrane of liposomes. Because of these characteristics, liposomes have been utilized in fields including medical services, cosmetics, food and so on. Thorough research into the use of liposome formulations for drug delivery systems (DDS) is recently ongoing. Also the lipid bilayer membrane of liposomes is combined of proteins or peptides, and thus evaluation of the actions thereof is being studied.

Examples of methods of manufacturing liposomes are known to include vortex treatment, ultrasonic treatment, reverse-phase evaporation, ethanol injection, extrusion, surfactant treatment, and static hydration (Non-Patent documents 1, 2). These manufacturing methods are selected between depending on the structure of liposomes. Ultrasonic treatment has been used to date from the beginning of the research into liposomes. Hence, a liposome manufacturing device is being developed using this method (Patent document 1). Also, a liposome manufacturing device using a super-critical fluid is being developed (Patent document 2).

### CITED REFERENCE

Patent Document

Patent document 1: Japanese Unexamined Patent Publication No. Hei. 4-293537

Patent document 2: Japanese Unexamined Patent Publication No. 2005-162702

Non-Patent Document

Non-Patent document 1: Hiroshi TERADA, Tetsuro YOSHIMURA, [Liposome test manual in life science], Springer-Verlag Tokyo (1992)

Non-Patent document 2: V. P. Torchilin, V. Weissig, “Liposomes”, Oxford University Press (2003)

### SUMMARY OF INVENTION

### Technical Problem

The liposome manufacturing device that uses ultrasonic treatment disclosed in Patent document 1 is problematic because only a small amount of the solution to be ultrasonically treated one time can be used if the device cannot be automatically controlled. The liposome manufacturing device using a super-critical fluid disclosed in Patent document 2 is problematic because it needs a vessel which endures high pressure and so is bulky.

Because of such problems, the formulation of liposomes is being currently mainly performed by manual works. Liposomes are manually formulated in such a manner that the lipid of liposomes is first dissolved in an organic solvent thus preparing a lipid-organic solvent solution, which is then placed in a flask, the inside of the flask is decompressed while rotating the flask, so that the organic solvent is gradually gasified, thereby forming a thin lipid film on the inner wall of the flask. The reason why the formulation procedures are based on the above sequence is that the formation of a uniform thin lipid film is regarded as important in order to formulate liposomes having good quality. Hence, a round-bottom flask having as large a bottom area as possible is used. In the above methods, in order to widely spread the thin lipid film, a large amount of organic solvent is used, which is environmentally unfriendly.

Manufacturing of the thin lipid film using the above methods requires much effort and time. So, a large work force is necessary to manufacture many kinds of or large amounts of liposomes.

Accordingly, the present invention has been made keeping in mind the problems encountered in the related art, and is intended to provide a device for rapidly and efficiently formulating large amounts of many kinds of liposomes using a small amount of organic solvent under computer-based automatic control. The present inventors manufactured a relatively small mechanical device equipped with a cylindrical reaction vessel and an eccentric motor. This device is further combined with an ultrasonic processor, thus completing an automatic liposome manufacturing device under computer-based automatic control. This device enables a variety of liposomes to be rapidly and efficiently manufactured.

### Solution to Problem

Intensive and thorough research into manufacturing liposomes, carried out by the present inventors, led to the development of a device which is configured such that a cylindrical reaction vessel, an eccentric motor and an ultrasonic processor are controlled by a computer thus enabling the manufacturing of a variety of liposomes such as MLV (Multi-Lamellar Vesicles), LUV (Large Unilamellar Vesicles), SUV (Small Unilamellar Vesicles), GUV (Giant Unilamellar Vesicles) and so on.

Thereby, an automatic multifunctional liposome manufacturing device according to a first embodiment of the present invention has the following features.

Specifically, this device comprises a cylindrical reaction vessel,

an eccentric motor for generating a vortex flow in a solution stored in the reaction space of the reaction vessel,

a heater for heating the reaction vessel to a predetermined temperature,

an aqueous solution line provided to the reaction vessel so as to introduce an aqueous solution into the reaction space,

a first bottle provided on the other end of the aqueous solution line so as to store the aqueous solution,

a first pump for transferring the aqueous solution into the reaction space via the aqueous solution line from the first bottle,

an inert gas line provided to the reaction vessel so as to introduce an inert gas into the reaction space,

a decompression line for decompressing the reaction space,

a vacuum pump for decompressing the reaction space via the decompression line,

a lipid line provided to the reaction vessel so as to introduce an organic solvent having lipid dissolved therein into the reaction space,

a second bottle provided on the other end of the lipid line so as to store the organic solvent,

a second pump for transferring the organic solvent into the reaction space via the lipid line from the second bottle,

an organic solvent recovery unit for recovering the organic solvent,

and a computer for controlling the eccentric motor, the heater, the first pump and the second pump, wherein under computer-based control, the inert gas is introduced into the reaction vessel, and the eccentric motor is driven so that a vortex flow is generated in the organic solvent having lipid dissolved therein that was put in the reaction space, and the reaction space is decompressed and thus the organic solvent is gasified in the reaction space and recovered by the organic solvent recovery unit, thereby forming a thin lipid film on the inner wall of the reaction vessel, and then the inert gas is introduced into the reaction space, and the aqueous solution is introduced into the reaction vessel, the eccentric motor is driven to thus generate a vortex flow in the aqueous solution, thereby manufacturing liposomes from the thin lipid film and the aqueous solution.

In the present invention, the following features are preferably provided.

Specifically, the aqueous solution line branches into a plurality of lines at the end opposite the reaction vessel, and an end of each of the plurality of lines is equipped with a water-based bottle for storing a solvent composed mainly of water and a water-based pump for transferring the solvent into the reaction space via the aqueous solution line from the water-based bottle, and each water-based pump is controllable by the computer, and the lipid line branches into a plurality of lines at the end opposite the reaction vessel, and an end of each of the plurality of lines is equipped with an organic-based bottle for storing a solvent composed mainly of an organic solvent and an organic-based pump for transferring the solvent into the reaction space via the lipid line from the organic-based bottle, and each organic-based pump is controllable by the computer.

Also the following features are preferably provided.

Specifically, a solution transfer line for sucking the aqueous solution stored in the reaction space so as to transfer it, and a transfer pump controlled by the computer so as to suck the aqueous solution are provided, and the other end of the solution transfer line is equipped with the ultrasonic processor controlled by the computer so as to irradiate the aqueous solution with ultrasonic waves.

In the present invention, the inert gas line and the decompression line may became the same line by using a three (or more)-way cork.

According to the present invention, under computer-based control, the organic solvent having lipid dissolved therein (lipid-organic solvent solution) is stored in the reaction space, and the eccentric motor is driven, so that a vortex flow is generated in the lipid-organic solvent solution, and the organic solvent is gasified, thereby forming the thin lipid film on the inner wall of the reaction vessel.

In conventional methods, an organic solvent having lipid dissolved therein is placed in a round-bottom flask, and the organic solvent is gradually removed under a nitrogen stream or under reduced pressure, so that the thin lipid film forms on the bottom of the flask, undesirably lengthening the process time. Meanwhile, the present inventors adopted the cylindrical vessel instead of the bulky round-bottom flask. So, eccentric rotation is imparted to the cylindrical vessel using the eccentric motor attached to the bottom of the cylindrical vessel, thereby generating a vortex flow in the lipid-organic solvent solution inside the vessel. While this vortex flow is being maintained, the vessel and the inside of a system are decompressed using the vacuum pump, and the organic solvent is thus gasified and removed, successfully forming the thin lipid film.

When a vortex flow is generated in the solution of the reaction space of the cylindrical vessel in this way by driving the eccentric motor, the solution develops upwards along the inner wall of the vessel. In this state, when the reaction space is decompressed, the organic solvent may be rapidly removed because of the large surface area of the solution. Also, the thin lipid film which is spread widely along the inner wall of the cylindrical vessel may be formed. The aqueous solution such as a buffer is placed in the cylindrical vessel on which the thin lipid film was formed, and the eccentric motor is driven, thus generating a vortex flow in the aqueous solution of the reaction space, thereby hydrating and stripping the thin lipid film, ultimately manufacturing liposomes.

The operating conditions of the device including the lipid composition, the solvent composition, the aqueous solution composition, and the volumes of the cylindrical vessel, the temperature, and the eccentric motor driving conditions (i.e. vortex flow properties) are adjusted, and thereby a variety of liposomes can be produced. Conventionally, there was known a system for decompressing the reaction space to remove an organic solvent while the reaction vessel is shaken in back and forth or right and left directions. In the present invention, the use of the eccentric motor is adopted because the organic solvent is removed while the solution develops well along the inner wall of the vessel, and hence the organic solvent of the inner space can be prevented from bumping and can also be rapidly removed. The eccentric motor may be used for both the removal of the organic solvent and the production of liposomes, thus simplifying the structure of the manufacturing device.

Because there is no need to detach the reaction vessel when liposomes are being produced, it is possible to completely automate the manufacturing device with the computer. Continuous operation of the device enables liposomes to be mass produced. Furthermore, because the thin lipid film is produced while generating a vortex flow, the solvent develops well along the inner wall of the vessel. Hence, a small amount of the organic solvent can be used, and thus loads on the environment are smaller compared to when using conventional manual work.

Almost all the processes for manufacturing liposomes take place in a closed system, and thus decompression, deoxygenation, nitrogen substitution, and sterilization may be carried out in the reaction space. So, concerns about the mixing (contamination) of microorganisms are reduced, and thus the present invention can be applied to the manufacture of medicines.

According to this construction, the lipid line and the aqueous solution line are separated, thus facilitating cleaning of respective lines.

The liposomes are a variety of liposomes such as MLV, LUV, SUV, GUV and so on as mentioned above, and include (1) liposomes which enclose a water-soluble drug, antibody, enzyme, gene, etc. in an aqueous phase surrounded with a lipid bilayer membrane, (2) liposomes in which an oil-soluble drug is enclosed in the lipid bilayer membrane, (3) liposomes in which functional protein, peptide, biopolymer or the like is held in the membrane using bonding, labeling or perforation, and (4) non-enclosed liposomes in which any material is not enclosed. Also, the liposomes include (5) a liposome vaccine in which protein, peptide, biopolymer or the like for an antigen is held in the membrane. The liposome vaccine inoculates the living body so that an antibody adapted for the antigen held in the membrane is manufactured, resulting in a vaccine. As used herein, the liposomes are multifunctional liposomes having any one among the above examples.

When the plurality of water-based bottles and the plurality of organic-based bottles are provided, a plurality of water-based solvents and a plurality of organic-based solvents may be prepared, and a variety of options for manufacturing liposomes are created, thus enabling a variety of liposomes to be manufactured. Because the lines for the water-based solvent and the organic-based solvent are separated, cleaning of respective lines becomes easy.

The term “ultrasonic processor” means a device for irradiating a liquid with ultrasonic waves, so that a material (e.g. bacteria, virus, etc.) included in the liquid is dispersed and broken, or liposomes (in particular, SUV) are formed with lipids. According to the present invention, automatic control is possible with the computer, and thus, an antigen such as bacteria, virus or the like is irradiated with ultrasonic waves, and a lipid solution for forming liposomes may be added. Generally, the period of time required until a material (e.g. protein) dispersed by ultrasonic irradiation is re-agglutinated after stopping ultrasonic irradiation is on the order of mm/sec. Hence, when a liposome solution is added to the material irradiated with ultrasonic waves according to conventional manual works, the probability of initiating (or terminating) re-agglutination upon addition of the liposome solution may increase, making it impossible to efficiently combine the dispersed material with liposomes. However, in the present invention, liposomes may be introduced in a state of the material being dispersed, and thus liposomes (third liposomes) having such a dispersion state can be formulated.

The liposome vaccine is used to, by pre-inoculating an antigen such as a phatogenic organism (i.e. bacteria, virus, etc.) causing specific infection into animals for example a human, dogs, cats, fish and so on, improve immunity so as to prevent the infection, wherein liposomes transport the antigen as a carrier. Such liposomes enable the antigen to stay in the inner aqueous phase thereof or to be charged in the lipid bilayer membrane or to be bound to the surface of the lipid bilayer membrane, and are thus considered to be immune cells to a variety of antigens having original forms as possible. Hence, in the liposome vaccine according to the present invention, better immune effects can be expected than for conventional vaccines.

A method of manufacturing liposomes according to the present invention includes manufacturing liposomes using the automatic multifunctional liposome manufacturing device as above, and has the following steps of (1) introducing an inert gas into the reaction space of a reaction vessel, and inside the reaction space, while generating a vortex flow in an organic solvent having lipid dissolved therein that was put in the reaction space, decompressing the reaction space, so that the organic solvent is gasified in the reaction space, thus forming a thin lipid film on the inner wall of the reaction vessel, and (2) introducing the inert gas into the reaction space, adding an aqueous liquid to the thin lipid film, and generating a vortex flow in the aqueous liquid inside the reaction space, thus formulating first liposomes.

Also a method of manufacturing liposomes according to the present invention includes manufacturing liposomes using the automatic multifunctional liposome manufacturing device including an ultrasonic processor, and has the following steps of (1) introducing an inert gas into the reaction space of a reaction vessel, and inside the reaction space, while generating a vortex flow in an organic solvent having lipid dissolved therein that was put in the reaction space, decompressing the reaction space, so that the organic solvent is gasified in the reaction space, thus forming a thin lipid film on the inner wall of the reaction vessel, (2) introducing the inert gas into the reaction space, adding an aqueous liquid to the thin lipid film, and generating a vortex flow in the aqueous liquid inside the reaction space, thus formulating first liposomes, and (3) irradiating the first liposomes with ultrasonic waves using the ultrasonic processor, thus formulating second liposomes.

Also a method of manufacturing liposomes according to the present invention includes manufacturing liposomes using the automatic multifunctional liposome manufacturing device including an ultrasonic processor, and has the following steps of (1) introducing an inert gas into the reaction space of a reaction vessel, and inside the reaction space, while generating a vortex flow in an organic solvent having lipid dissolved therein that was put in the reaction space, decompressing the reaction space, so that the organic solvent is gasified in the reaction space, thus forming a thin lipid film on the inner wall of the reaction vessel, (2) introducing the inert gas into the reaction space, adding an aqueous liquid to the thin lipid film, and generating a vortex flow in the aqueous liquid inside the reaction space, thus formulating first liposomes, and (4) irradiating a suspension liquid with ultrasonic waves using the ultrasonic processor and adding the first liposomes to the ultrasonic processor, thus formulating third liposomes.

In addition, a method of manufacturing liposomes according to the present invention includes manufacturing fourth liposomes from first liposomes˜third liposomes using the automatic multifunctional liposome manufacturing device, and has the following step of (5) introducing an inert gas into the reaction space, and while generating a vortex flow in a suspension of any one kind of liposomes among first liposomes˜third liposomes, adding an aqueous liquid, thus formulating fourth liposomes inside the reaction space.

The term “aqueous liquid” means a buffer, or an aqueous solution in which a water-soluble material (a compound, gene, protein (antibody, enzyme, etc.), saccharides, polysaccharides, active ingredient, etc.) is dissolved. The term “suspension liquid” means a liquid in which a material (a compound, active ingredient, polysaccharides, bacteria, virus, etc.) which is difficult to dissolve is suspended in water (including a buffer).

The term “first liposomes” means (i) MLV, LUV and GUV, (ii) MLV, LUV and GUV in which a water-soluble material is enclosed and wherein an oil-soluble material is enclosed in the membrane, (iii) liposome vaccine (MLV and LUV) in which an antigen is enclosed, and (iv) MLV, LUV and GUV the membrane surface of which is formulated with PEG·saccharide chains.

The term “second liposomes” means (i) SUV, (ii) SUV in which a water-soluble material is enclosed and wherein an oil-soluble material is enclosed in the membrane, (iii) SUV in which an antigen is enclosed, and (iv) SUV the membrane surface of which is formulated with PEG·saccharide chains.

The term “third liposomes” means (i) liposomes which are efficiently manufactured, (ii) liposome vaccine representing a bacteria and virus surface antigen, and (iii) liposomes in which a material having high cohesion (e.g. macromolecular protein, polysaccharides, virus derived antigens, membrane protein, etc.) is ultrasonically crushed and enclosed.

The term “fourth liposomes” means reconfigured liposomes, (i) protein- or peptide-bound MLV, SUV, LUV and GUV, (ii) antigen-bound liposome vaccine (MLV, SUV and LUV), and (iii) recombinant proteoliposomes (baculovirus fused MLV, SUV, LUV and GUV).

### Advantageous Effects of Invention

According to the present invention, an automatic multifunctional liposome manufacturing device includes an eccentric motor, an ultrasonic processor, and a computer for automatic control of the operation thereof. By continuously operating this device, large amounts of many kinds of liposomes can be rapidly and accurately manufactured.

### DESCRIPTION OF DRAWINGS

FIG. 1 is a schematic view showing the construction of an automatic multifunctional liposome manufacturing device;

FIG. 2 is a schematic view showing the control construction of a computer;

FIG. 3 is a perspective view showing the manufacturing device;

FIG. 4 is a front view showing the manufacturing device;

FIG. 5 is a side view showing the manufacturing device;

FIG. 6 is a top plan view showing the manufacturing device;

FIG. 7 is a view showing the back surface of the manufacturing device from which a cover thereof is removed;

FIG. 8 is a perspective view showing the frame and the heater of the manufacturing device;

FIG. 9 is a side view around the heater;

FIG. 10 is a side view showing an ultrasonic processor and an eccentric motor;

FIG. 11 is a perspective view showing a shaking maintenance unit;

FIG. 12 is a back view showing the shaking maintenance unit;

FIG. 13 is a side view showing the shaking maintenance unit;

FIG. 14 is a photograph showing the manufacturing device;

FIG. 15 is a flowchart showing a manufacturing process of liposomes (MLV: first liposomes);

FIG. 16 is a flowchart showing a manufacturing process of liposomes (SUV: second liposomes) using ultrasonic treatment;

FIG. 17 is a flowchart showing a manufacturing process of liposome vaccine (third liposomes) using inactivated virus; and

FIG. 18 is a flowchart showing a manufacturing process of peptide-bound liposomes (fourth liposomes) using an eccentric motor.

### DESCRIPTION OF EMBODIMENTS

Hereinafter, embodiments of the present invention will be described with reference to the drawings. The technical scope of the present invention is not limited to such embodiments, but may be embodied in various forms without departing the purport of the present invention. Also the technical scope of the present invention extends to equivalent ranges.

<Automatic Multifunctional Liposome Manufacturing Device>

1. Connected Construction of Automatic Multifunctional Liposome Manufacturing Device

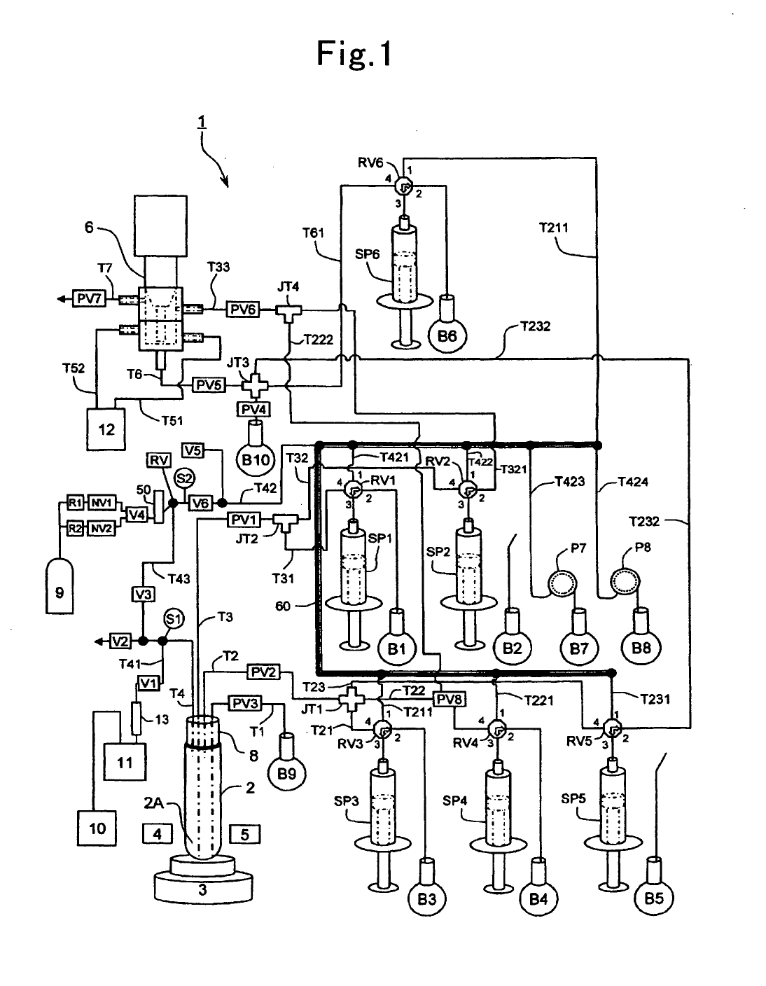

With reference to FIG. 1, the construction of an automatic multifunctional liposome manufacturing device (an automatic liposome vaccine manufacturing device) 1 is described, which is simply referred to as a manufacturing device 1 below. The manufacturing device 1 is able to automatically perform operations including forming a thin film of lipid dissolved in chloroform and then manufacturing liposomes from the thin lipid film in a predetermined aqueous solution (e.g. an appropriate buffer), by means of a computer.

The manufacturing device 1 comprises a cylindrical reaction vessel 2 having a reaction space 2A, an eccentric motor 3 with an eccentric shaft for generating a vortex flow in a solution stored in the reaction space 2A inside the reaction space 2A, a heater 4 for spraying hot air or cold air into the reaction vessel 2, and a temperature sensor 5 for measuring the temperature of the reaction vessel 2. The top of the reaction vessel 2 is closed by a cap 8, and four lines T1˜T4 are formed through the cap 8. For the sake of description, the lines have appropriate names, but respective lines do not perform only functions mentioned in such names, but may execute other functions based on the control of the computer 15.

The ends of two lines T1, T3 extend to the bottom of the reaction vessel 2, in which T1 designates a cleaning solution recovery line for recovering a cleaning solution and T3 designates a solution recovery line for carrying a liposome solution into an ultrasonic processor 6. The ends of the other two lines T2, T4 are positioned at the upper portion of the reaction vessel 2, in which T2 designates a transport line for dropping an antigen or a buffer into the reaction vessel 2 and T4 designates a gas handling line for blowing an inert gas into the reaction vessel 2 or connecting the reaction vessel 2 with a vacuum unit so that the reaction vessel 2 is decompressed. A pinch valve PV3 is provided on the route of the line T1, and the other end thereof is connected to a cleaning solution recovery bottle B9. Also a pinch valve PV2 and a four-way connector JT1 are provided on the route of the line T2, and three lines T21˜T23 branch off from such a connector JT1.

The branched line T21 is provided with a rotary valve RV3. The manufacturing device 1 includes six rotary valves RV1˜RV6. Respective rotary valves RV1˜RV6 have rotary spools provided therein, and the rotary spools are rotated under control of a computer 15, whereby any path of positions of 1-2, 2-3, 3-4, and 4-1 among Nos. 1˜4 around the rotary valves RV1˜RV6 may be connected. In FIG. 1, all the rotary valves RV1˜RV6 are in a state of the path of a position of 1-2 being connected. The rotary valve RV3 is connected to a syringe pump SP3, a bottle B3, and a branched line T211.

The end of the branched line T22 is provided with a pinch valve PV8. The pinch valve PV8 is a three-way valve to which the lines from the rotary valve RV4 and the line from the three-way connector JT4 are connected. The rotary valve RV4 is connected to a syringe pump SP4, a bottle B4, and a branched line T221. The branched line T23 is provided with the rotary valve RV5 to which a syringe pump SP5 and two branched lines T231, T232 are connected.

The line T3 is provided with a pinch valve PV1, and a three-way connector JT2 is provided in front of it. Two lines T31, T32 branch off from this connector JT2. The branched line T31 is provided with the rotary valve RV1 to which a syringe pump SP1, a bottle B1 and a line T421 are connected.

The branched line T32 is provided with the rotary valve RV2 to which a syringe pump SP2, a bottle B2 and two lines T422, T321 are connected. The line T321 is connected to the three-way connector JT4 facing the ultrasonic processor 6. The connector JT4 is connected with a line T33 for supplying a solution to the ultrasonic processor 6 and a line T222 connected to the pinch valve PV8. The pinch valve PV6 is provided on the route of the line T33.

The line T4 is connected with a line T41 connected to a vacuum pump 10, an organic solvent recovery unit 11 and an air drying tube 13, and a line T43 connected to a gas bomb 9 for supplying nitrogen as an inert gas. Port valves V1, V3 are provided on the route of respective lines T41, T43. Also, a pressure sensor S1, and a port valve V2 for releasing internal pressure of the line to external air are provided on the route of the line T41.

The line guided from the gas bomb 9 branches into two branches which respectively pass through regulators R1, R2 and needle valves NV1, NV2 and are then connected to the port valve V4. The port valve V4 is connected with a line T42. Furthermore, this line T42 is sequentially connected to a flow meter 50, the line T43, a relief valve RV, a pressure gauge S2, and port valves V6, V5. The end of the line T42 is connected to a manifold 60. Also the manifold 60 is connected to, in addition to the line T42, lines T421, T422, T211, T221, T231, T211 from six rotary valves RV1˜RV6, and lines T423, T424 from two tube pumps P7, P8. The tube pumps P7, P8 are connected to the bottles B7, B8, respectively.

In order to maintain a sample at low temperature, the ultrasonic processor 6 is connected with a circulating water supply line T51 and a circulating water recovery line T52, which circulate cooling water from a circulatory unit 12, and also with sample supply and recovery lines T33, T6 for supplying and recovering a solution to be ultrasonically treated. Furthermore, a line T7 for releasing the pressure of gas and a pinch valve PV7 are connected to the ultrasonic processor 6 upon supply of the sample. The line T6 is provided with the pinch valve PV5 and the four-way connector JT3. Also the connector JT3 is connected to a line connected to a recovery bottle B10 having the pinch valve PV4, the branched line T232 connected to the rotary valve RV5, and the branched line T61 connected to the rotary valve RV6. The rotary valve RV6 is connected to the syringe pump SP6, the bottle B6, and the branched line T211.

In FIG. 1, because there is no need to use two bottles B2, B5 in the present embodiment, the connection of these bottles is removed. However, the bottles B2, B5 are respectively connected to the rotary valves RV2, RV5, and thus may perform inflow/outflow of the solution in response to the operation of the syringe pumps SP2, SP5, as necessary.

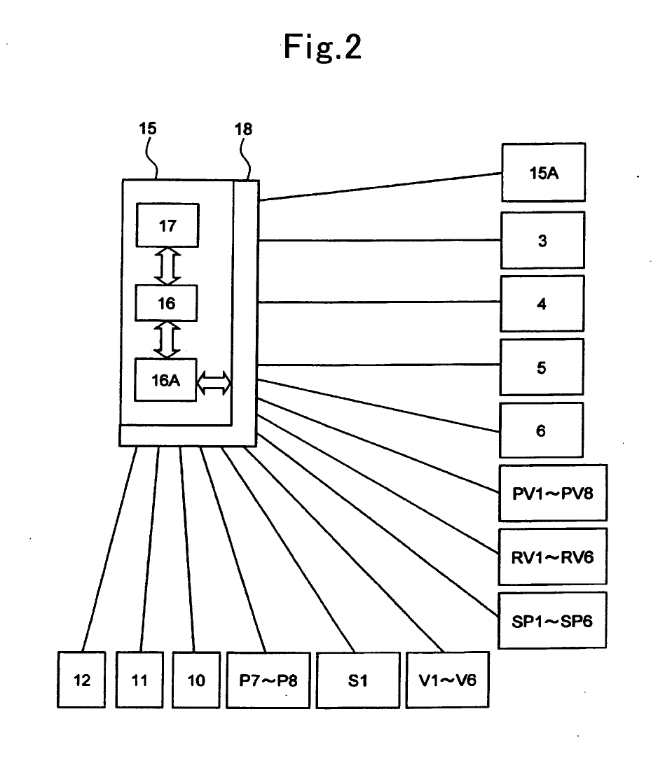

As shown in FIG. 2, the manufacturing device 1 includes the computer 15 comprising a liquid crystal unit 15A, a CPU 16, a control substrate 16A, a memory unit 17, and an I/O port 18. The computer 15 is known and its detailed description is omitted. The computer 15 is connected to the eccentric motor 3, the heater 4, the temperature sensor 5, the ultrasonic processor 6, the pinch valves PV1˜PV8, the rotary valves RV1˜RV6, the syringe pumps SP1 SP6, the port valves V1˜V6, the pressure sensor S1, the tube pumps P7˜P8, the vacuum pump 10, the organic solvent recovery unit 11, and the circulatory unit 12, and is able to input signals from the respective units and/or output signals to the respective units and thus may control the operation of respective units depending on the preset programs. Also the liquid crystal unit 15A includes the pressure sensor, and a predetermined image may be displayed in accordance with a predetermined program, and appropriate parameters may be input.

2. Mechanical Construction of Automatic Multifunctional Liposome Manufacturing Device

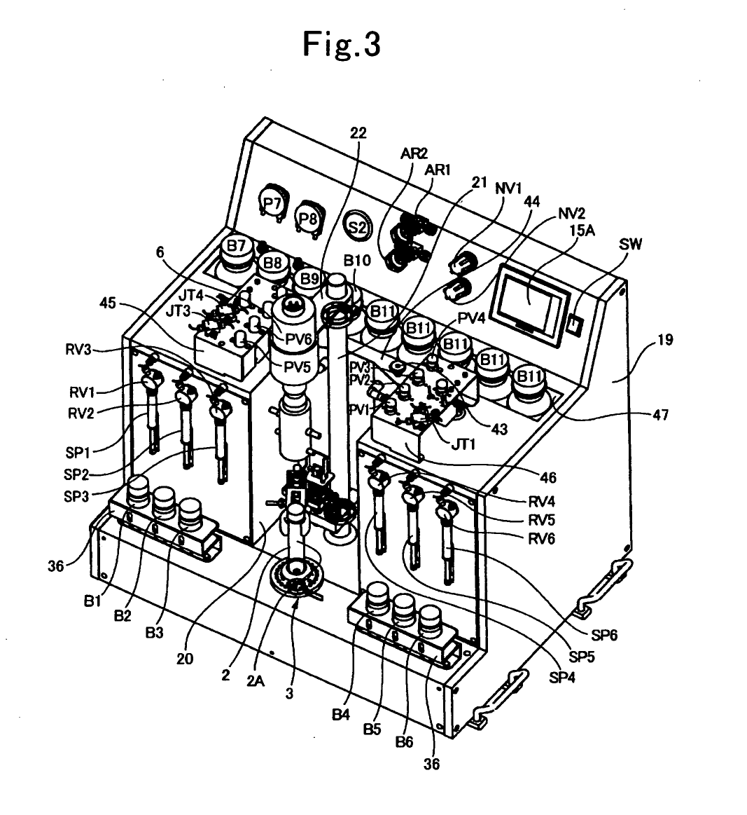

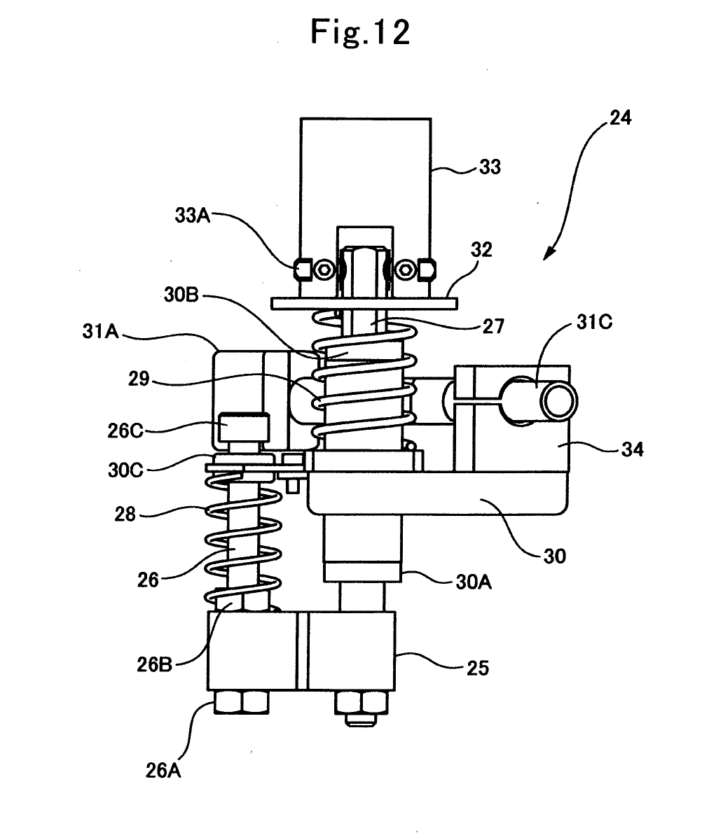

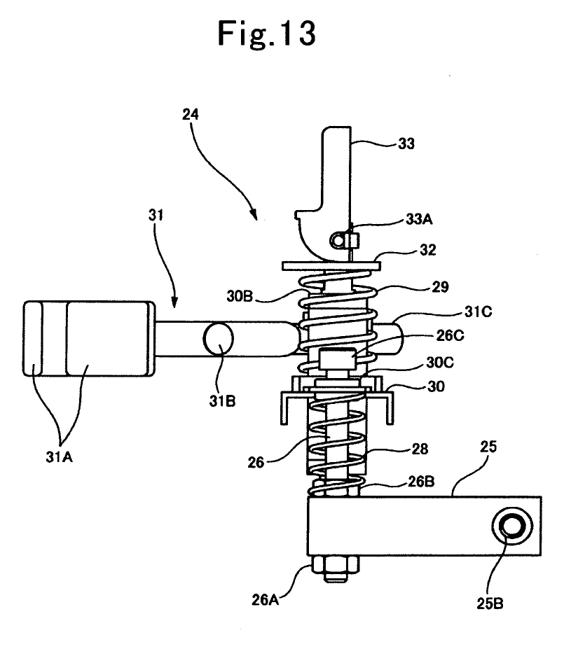

With reference to FIGS. 3 to 13, the mechanical construction of the manufacturing device 1 is described. Furthermore, for the sake of simplicity, only parts of lines are depicted.

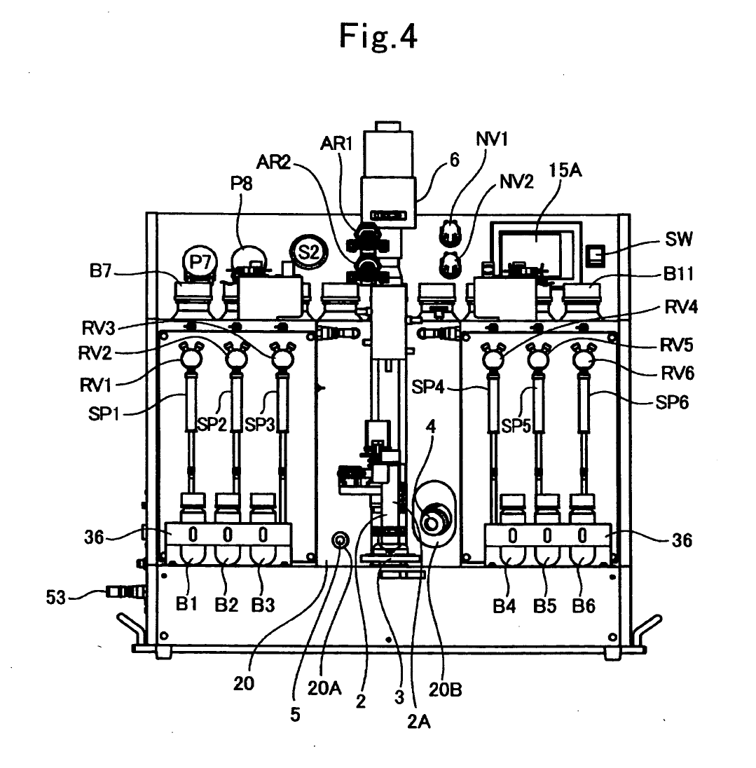

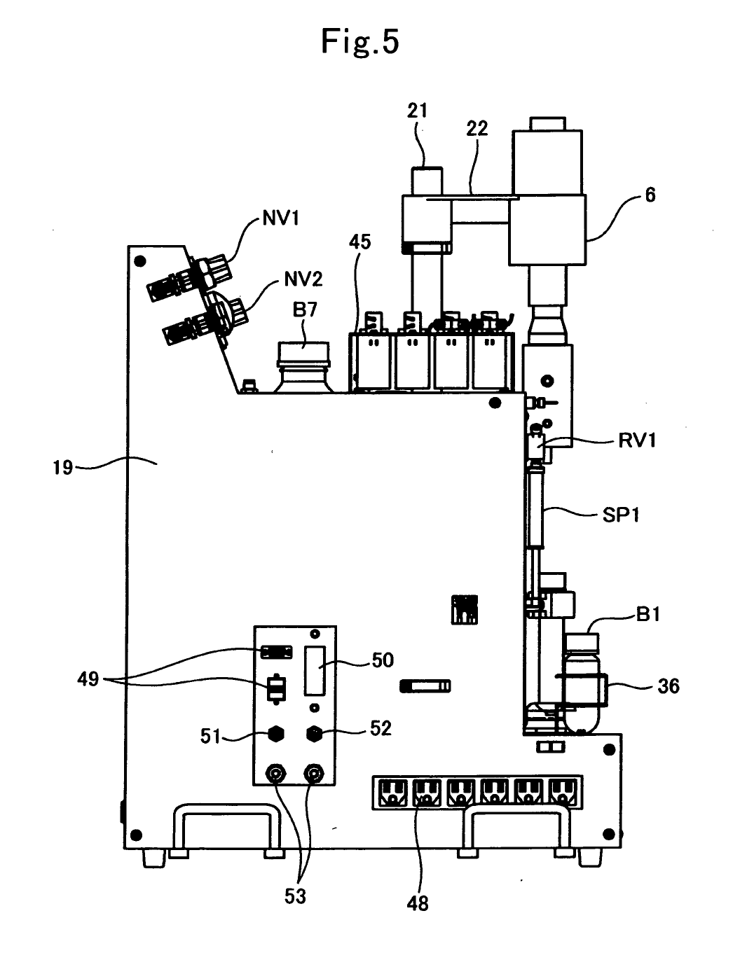

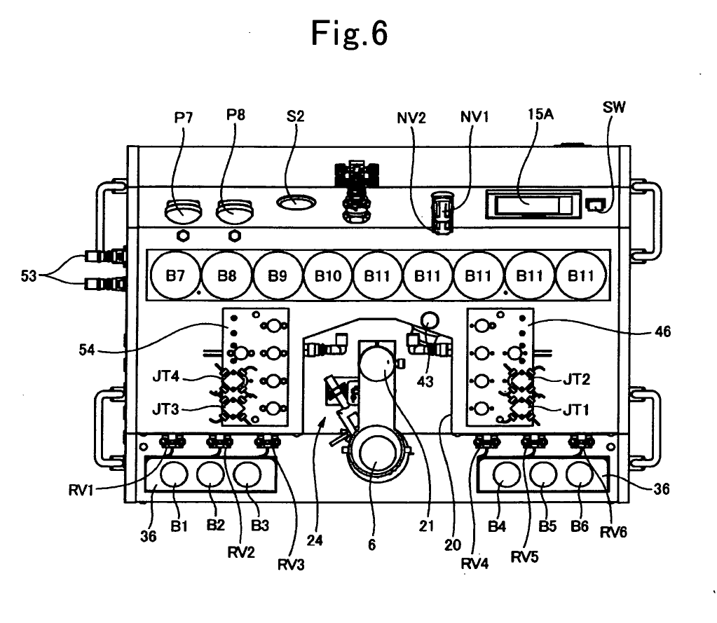

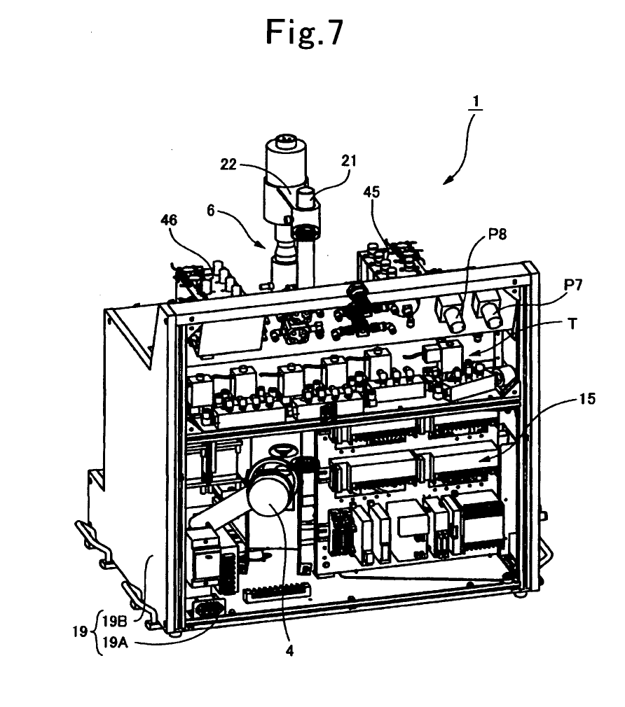

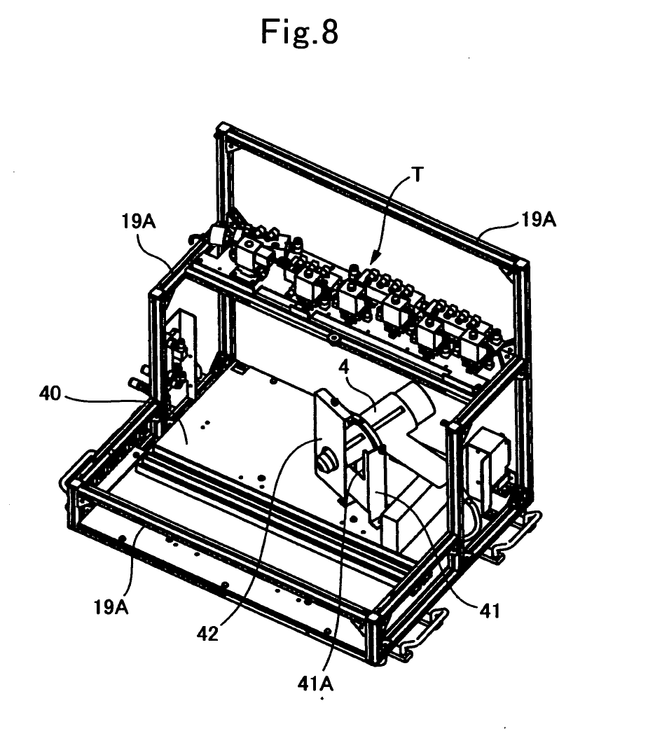

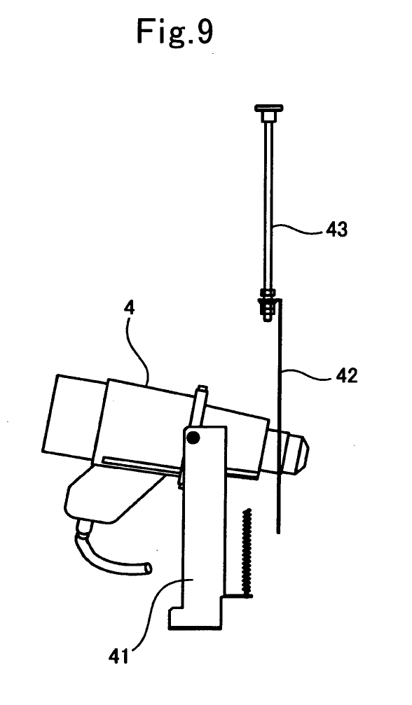

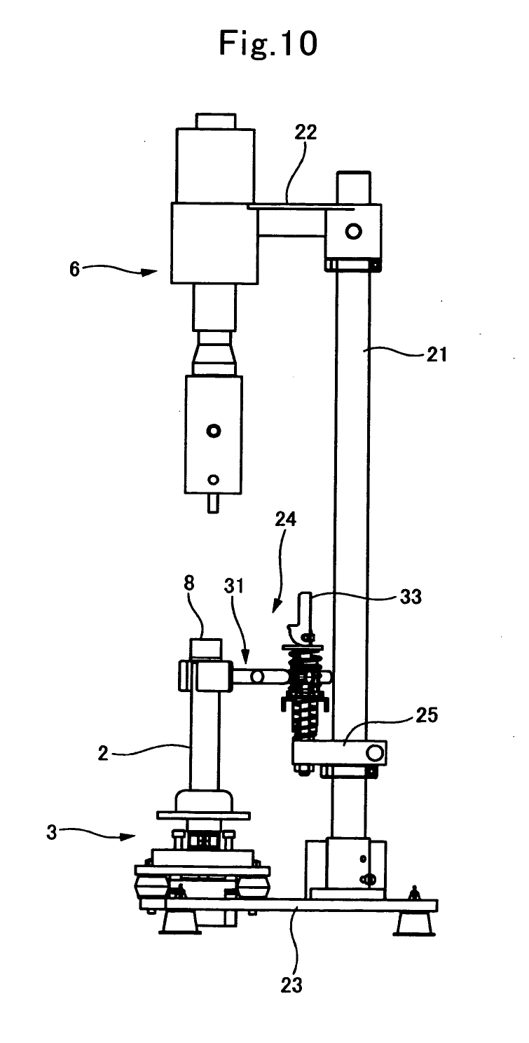

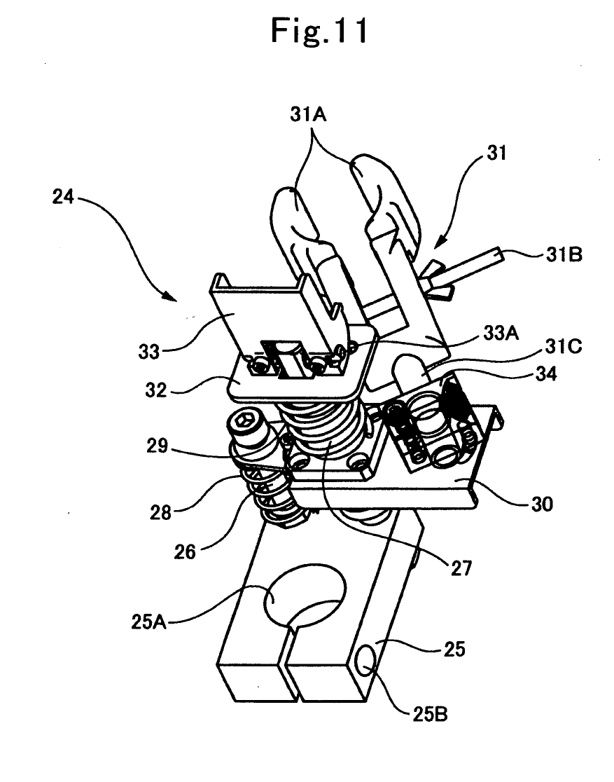

The manufacturing device 1 is depicted in the form of a perspective view in FIG. 3, a front view in FIG. 4, a side view in FIG. 5, a top plan view in FIG. 6, and a back view (which shows the inside of the device without a cover) in FIG. 7, and the frame and the heater thereof are shown in FIG. 8. FIG. 9 is a side view around the heater, FIG. 10 is a side view of the ultrasonic processor and the eccentric motor, and FIGS. 11 to 13 are a perspective view, a back view and a side view of a shaking maintenance unit, respectively.

As shown in FIGS. 7 and 8, a main body 19 has a frame 19A famed of bar-type materials (e.g. using a metal such as aluminum), and the frame 19A is covered with a surface cover 19B made of a sheet (e.g. stainless steel). As shown in FIGS. 3 and 4, the computer 15, the lines T and so on are received in the main body 19. A recess 20 is formed in a longitudinal direction at the center of the main body 19. A support column 21 is set up in a vertical direction in the recess 20, and a support member 22 is provided forward at the upper portion of the support column, in which the ultrasonic processor 6 is mounted to the support member. The lower end of the support column 21 is mounted to a support 23 provided in a horizontal direction, and the eccentric motor 3 is fixed in front of the support 23 (left side in FIG. 10).

As shown in FIGS. 10 to 13, the shaking maintenance unit 24 is provided in the vicinity of the lower portion of the support column 21. The shaking maintenance unit 24 functions to support the shaking of the reaction vessel 2. The shaking maintenance unit 24 comprises a support part 25 protruding forward horizontally from the support column 21, two column parts 26, 27 assembled in holes (not shown) perforating the support part 25 in up and down directions, springs 28, 29 wound loosely around both the column parts 26, 27, a connection sheet 30 that connects both the column parts 26, 27 and protrudes laterally from the column part 27, and a vessel bracket 31 protruding obliquely from the connection sheet 30. The support part 25 includes a mounting hole 25A which perforates it in up and down directions and is opened at a rear center thereof, into which the support column 21 is inserted. A screw fastening hole 25B that perforates the support part 25 in a transverse direction is provided at the rear of the mounting hole 25A, and a screw which is not shown is assembled, so that the support part 25 may be fixed at a predetermined position of the support column 21. The short column part 26 is assembled with nuts 26A, 26B. The assembly of the nuts 26A, 26B enables the fixing of the column part 26 to the support part 25. Also an upper protrusion 26C is formed at the upper end of the column part 26. The connection sheet 30 has the hole that perforates the column part 26, and a rubber member 30C is mounted around this hole. When it is intended to move the connection sheet 30 to a level exceeding the norm, the column part 26 comes into elastic contact with the rubber member 30C, thus controlling the movement and stopping it from exceeding the norm.

Around the long column part 27 assembled in the connection sheet 30, cylindrical stoppers 30A, 30B are formed to protrude in up and down directions. The spring 29 is mounted above the connection sheet 30 in the column part 27, and a shaking control plate 32 is assembled on this spring. A cam lever 33 is axially fixed on the shaking control plate 32 by a shaft 33A that perforates the column part 27 laterally. The spring 29 is pressed by a predetermined force and fixed between the connection sheet 30 and the shaking control plate 32. When the reaction vessel 2 begins to be shaken at the level where the norm is exceeded, respective stoppers 30A, 30B come into contact with the upper surface of the support part 25 or the lower surface of the shaking control plate 32. So, the shaking of the reaction vessel 2 is controlled to within a predetermined range.

The vessel bracket 31 is provided with a bifurcated clamp 31A for holding the reaction vessel 2, a fastener 31B installed at the base of the bifurcated clamp 31A so as to fasten the reaction vessel 2 by a predetermined force, and a shaft 31C extending backward from the base of the clamp 31A. The fastener 31B includes a shaft member and a screw member, and the screw member is tightened to thereby fix the bifurcated clamp 31A using an appropriate amount of force. The portion of the clamp 31A in contact with the reaction vessel 2 is covered with a member (e.g. silicone rubber) having appropriate elasticity. The shaft 31C is inserted through a shaft holder 34 formed on the connection sheet 30 and is thus fixed. By the action of both the springs 28, 29, the shaking maintenance unit 24 enables the reaction vessel 2 to be uniformly shaken within a predetermined range by driving the eccentric motor 3.

As shown in FIGS. 3 and 4, the rotary valves RV1˜RV6 and the syringe pumps SP1˜SP6 are provided at both sides of the recess 20 over the entire surface of the main body 19. Also the bottles B1˜B6 are provided and are fitted into bottle holders 36 below these valves and pumps.

At the bottom of the inner wall of the recess 20, there are a sensor hole 20A for mounting the temperature sensor 5 and a heater hole 20B for mounting the heater 4. As shown in FIGS. 7 to 9, the heater 4 is provided in the form of a dryer. The heater 4 sprays hot air toward the lower end of the reaction vessel 2, thereby increasing the temperature of the reaction space 2A to a predetermined temperature. The heater 4 is assembled to an immobile sheet 41 attached to the lower sheet 40 of the main body 19. A C-shaped concave part 41A which opens upwards is provided at the upper surface of the immobile sheet 41. The heater 4 is mounted to the concave part 41A, and the heater 4 is fixed with screws at both ends of the immobile sheet 41 forming the concave part 41A. The front end of the heater 4 is inserted through a protective sheet 42. A bar-type bracket 43 is provided at the upper end of the protective sheet 42, and the upper portion of the bracket 43 is fixed to a sheet 44 installed at the upper end of the recess 20 and thus positioned (FIG. 3).

The rotary valves RV1˜RV6, the pinch valves PV1 PV8, and valve complexes 45, 46 each comprising a bundle of various lines are provided at both sides of the recess 20 at the upper surface of the main body 19. Also in the main body, a depression 47 is formed for bottles in a row, which includes bottles B7˜B10 and a preliminary bottle B11. The upper portion of the main body 19 is formed to protrude forward, and the tube pumps P7, P8 and the pressure gauge S2 are sequentially mounted from the left side of the drawing. Also, the liquid crystal unit 15A and the main switch SW of the manufacturing device 1 are mounted at the right side of the drawing.

As shown in FIG. 5, sockets 48 for electrical connection, connectors 49 for connection with an external electronic device, a nitrogen gas flow meter 50, a nitrogen gas inlet 51, a vacuum pump connector 52, and a cooling water inlet and outlet 53 are provided at the left side of the main body 19.

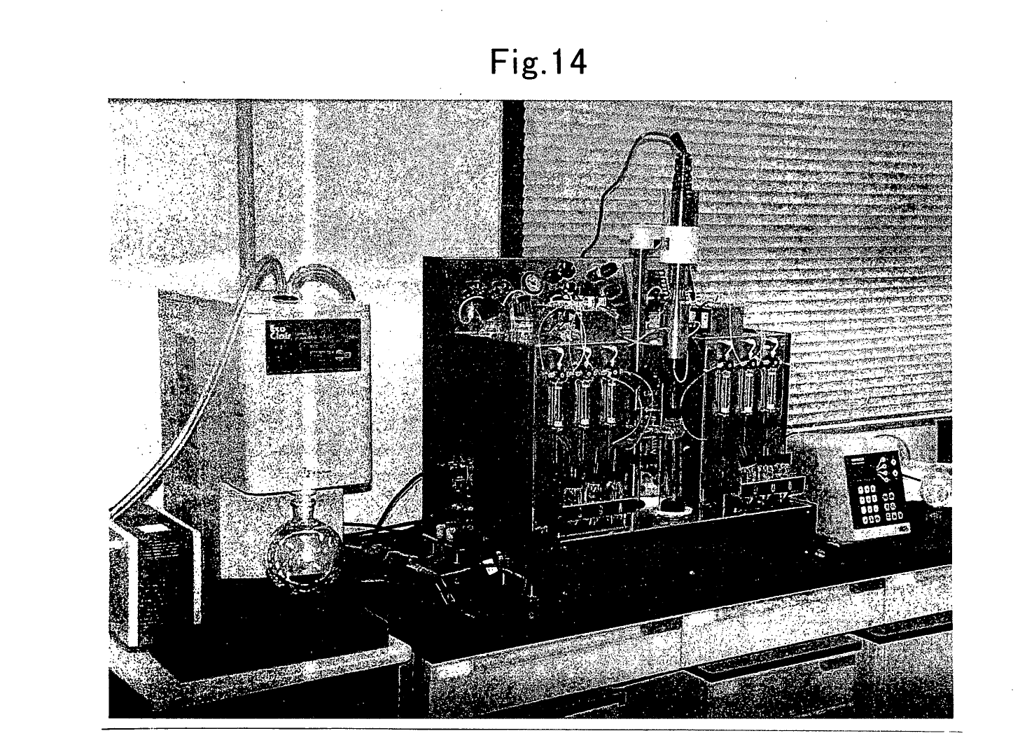

The actual photograph of the manufacturing device thus constructed is shown in FIG. 14. The main body thereof is shown at the center, the organic solvent recovery unit at the left side and the ultrasonic processor control unit at the right side.

<Manufacturing of Liposomes>

A method of manufacturing liposomes using the manufacturing device 1, and a control method (algorithm) are described.

1-1. Manufacturing of Liposomes (First Liposomes) Using Eccentric Motor

Using the eccentric motor 3, MLV which is a kind of first liposomes was formulated under automatic control of a computer 15.

A buffer and a lipid chloroform solution were previously stored in a bottle B4 (a first bottle) and a bottle B3 (a second bottle), respectively.

As the lipid chloroform solution, 2.5 ml of phospholipid (25 μmol dioleoylphosphatidyl choline and 25 μmol dioleoylphosphatidyl serine) dissolved in chloroform was used. As the buffer, 10.0 ml of 10 mM HEPES-NaOH/175 mM NaCl (pH 75) was used. After respective bottles were set, the liquid crystal unit 15A of the computer 15 was operated thus manufacturing MLV.

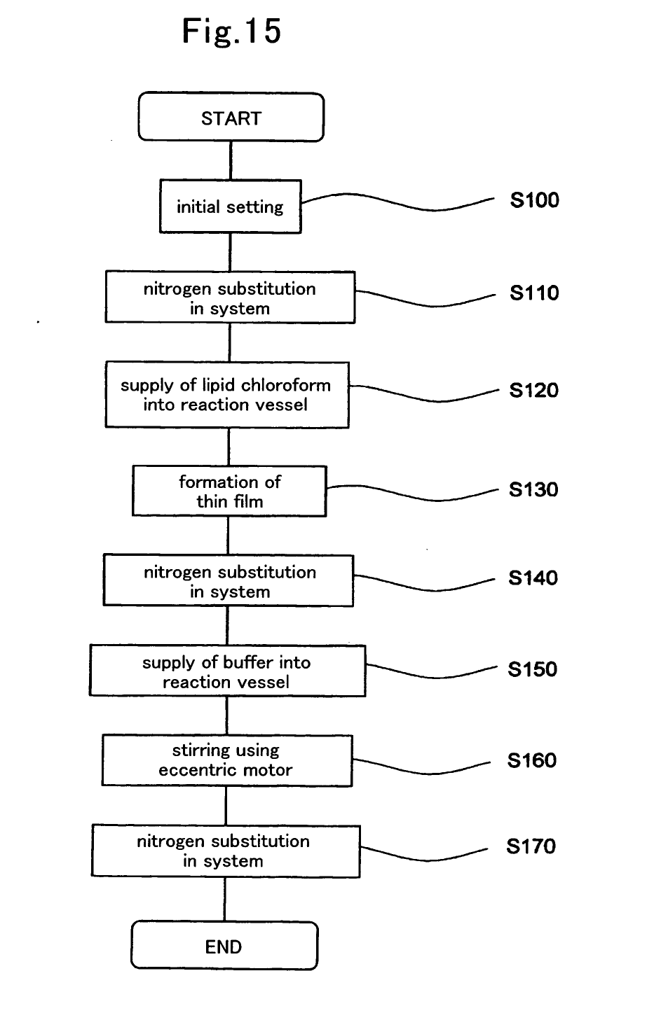

The MLV manufacturing algorithm is shown in FIG. 15. At the initial setting S100, the rotary valves RV1˜RV6 were rotated to a position of 4-1, the pinch valves PV1˜PV3 were closed, the port valves V1˜V3 were closed, and the port valves V4, V6 were opened, so that nitrogen was allowed to flow, and the regulators AR1=1 kPa, AR2=0.5 kPa were set, and the vacuum pump 10 and the organic solvent recovery unit 11 were set in a driving state, and the heater 4 and the eccentric motor 3 were set in a stationary state.

Next, nitrogen substitution in a system was performed (S110). In this step, the port valves V1, V2 and the pinch valve PV2 were opened, and nitrogen gas was allowed to flow over the route T42, the manifold 60, the routes T211, T221, T231, the routes T21, 22, 23, and the route T2 and the inside of the reaction vessel 2 was substituted with nitrogen.

Next, the lipid chloroform solution of the bottle B3 was supplied into the reaction vessel 2 (S120). In this step, the port valve V1 was closed, and the rotary valve R3 was rotated to a position of 2-3, and the syringe pump SP3 (a second pump) was operated for suction purposes, so that the lipid chloroform solution of the bottle B3 was sucked, after which the rotary valve RV3 was rotated to a position of 3-4, the pinch valve PV2 was opened, and the syringe pump SP3 was operated for discharge purposes, so that the lipid chloroform solution was supplied into the reaction vessel 2 via the line T21 (lipid line) and the line T2.

Next, thin film formation was performed (S130). In this step, the port valve V2 was closed and the port valve V1 was opened, so that the reaction space 2A was connected to the vacuum pump 10, and the eccentric motor 3 and the heater 4 were driven. Thereby, in the reaction space 2A at a high temperature, a vortex flow was generated in the lipid chloroform solution using the eccentric motor 3, and the reaction space 2A was decompressed using the vacuum pump 10, so that chloroform was gasified in the reaction space 2A, thus forming a thin lipid film on the inner wall of the reaction vessel 2.

Next, nitrogen substitution in a system was performed (S140). In this step, the heat source supply of the heater 4 was stopped, and cold air was added, in addition to the treatment as in S110, thereby cooling the reaction vessel 2.

Next, the buffer was supplied into the reaction vessel 2 (S150). In this step, the rotary valve RV4 was first rotated to a position of 2-3, the syringe pump SP4 was operated for suction purposes, so that the buffer was sucked from the bottle B4, after which the rotary valve RV4 was rotated to a position of 3-4, and the pinch valve PV8 and the pinch valve PV2 were opened, and then the syringe pump SP4 (a first pump) was operated for discharge purposes, and thus the buffer was supplied into the reaction vessel 2 through the line T211 (aqueous solution line) and the line T2. For this solution supply, the port valves V1, V3 were closed, and the port valve V2 was opened, thereby releasing the gas from the reaction space 2A by the solution supply.

Next, the eccentric motor 3 was driven, thus manufacturing MLV (S160). As such, in order to improve a thin lipid film stripping efficiency by the buffer, the eccentric motor 3 was alternately driven in forward/backward directions.

Finally, nitrogen substitution in a system was performed in the same manner as in S110. Thereby, the MLV having an average particle size of 534 nm could be manufactured using the buffer.

1-2. Manufacturing of LUV and GUV (First Liposomes)

Based on the manufacturing process of 1-1, LUV and GUV were manufactured. By appropriately changing the process sequence shown in FIG. 15, LUV having an average particle size of about 400 nm and GUV having an average particle size of about 20 μm could be manufactured.

1-3. Manufacturing of Water-Soluble Material-Enclosed Liposomes (First Liposomes)

This manufacturing process was performed in the same manner as in the manufacturing process of 1-1, with the exception that an enzyme (luciferase) dissolved was used instead of the buffer used at S150. Consequently, enzyme-enclosed MLV could be manufactured. Also in accordance with the manufacturing process of 1-2, enzyme-enclosed GUV could be manufactured.

In addition, the manufacturing process was performed in the same manner as above, using an active ingredient (Barbital), an antigen (green fluorescent protein), an antibody (anti-green fluorescent protein antibody), or nucleic acid (pBR322 vector) instead of the above enzyme, thereby manufacturing MLV and GUV in which the active ingredient, antigen, antibody or nucleic acid was enclosed.

1-4. Manufacturing of Liposomes (First Liposomes) Having Oil-Soluble Material Enclosed in Membrane Thereof

This manufacturing process was performed in the same manner as in the manufacturing process of 1-1, with the exception that a solution of lipid and oil-soluble material/chloroform was used instead of the lipid chloroform solution used at S120. As the oil-soluble material, α-tocopherol was used. Consequently, MLV having an oil-soluble material enclosed in the membrane thereof could be manufactured. In accordance with the manufacturing process of 1-2, LUV and GUV having an oil-soluble material enclosed in the membrane thereof could also be manufactured.

1-5. Use as Evaporator

In the manufacturing process of 1-1, an oil-soluble material/volatile organic solvent was used instead of the lipid chloroform solution used at S120. The oil-soluble material used was linoleic acid, and the volatile organic solvent was ethanol. After S100˜S140 were carried out, the volatile organic solvent was appropriately volatilized, thus concentrating the oil-soluble material. Thereby, the concentration of oil-soluble material could be performed in lieu of the formation of thin film. In this way, the manufacturing device 1 according to the present embodiment could be used as an evaporator.

2-1. Manufacturing of Liposomes (Second Liposomes) Using Ultrasonic Treatment

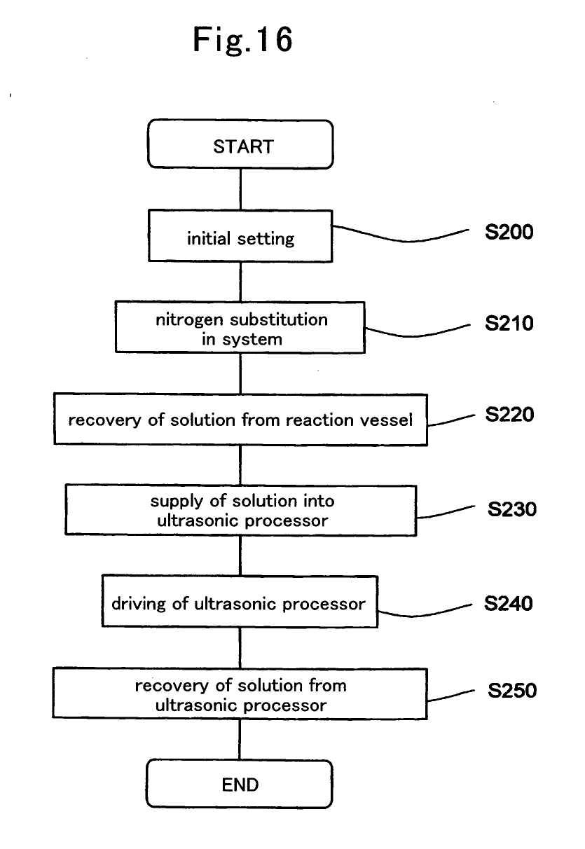

With reference to FIG. 16, a manufacturing process of SUV which is a kind of second liposomes is described. This process was initiated using buffer-MLV added into the reaction vessel 2, after S160. In addition, by appropriately changing the program set in the computer 15, the present process may be performed from an appropriate initial state (e.g. a state of MLV being recovered in any bottle).

At initial setting S200, ultrasonic treatment parameters (solution amount, size of ultrasonic processor, kind of ultrasonic chip, position of ultrasonic chip in vessel, etc.) were set. Also, the circulatory unit 12 was driven, whereby the ultrasonic processor 6 was cooled to a predetermined temperature (e.g. 0° C.).

At the next step (S210), nitrogen substitution in a system was performed. This treatment was carried out in the same manner as in the above step (S110).

Next, MLV was recovered from the reaction vessel 2 using a syringe pump SP2 (S220). In this step, while an inert gas from a gas bomb 9 was introduced into the reaction vessel 2 from the line T4, the syringe pump SP2 was operated for suction purposes. Specifically, only the port valves V3, V4 were opened so that a predetermined amount of nitrogen gas was introduced into the reaction vessel 2 from the line T4. Furthermore, the pinch valve PV1 was opened and the rotary valve RV2 was rotated to a position of 3-4, so that the syringe pump SP2 was operated for suction purposes.

Next, MLV was supplied into the ultrasonic processor 6 (S230). The rotary valve RV2 was rotated to a position of 2-3 and the pinch valves PV6, PV7 were opened, and thus the syringe pump SP2 was operated for discharge purposes, whereby MLV was supplied into the ultrasonic processor 6 through the lines T321, T33. Also in order to discharge the MLV in a system into the ultrasonic processor 6, compression of nitrogen gas was performed. For this compression, nitrogen gas was supplied into the ultrasonic processor 6 through the line T42, the manifold 60, the lines T321, T222, the joint JT4, and the line 133. Specifically, the port valves V4, V6 were opened (V3 was closed), the rotary valve RV2 was rotated to a position of 1-2 and the rotary valve RV4 was rotated to a position of 4-1 (the other rotary valves were in a state of the position 1 being closed), the pinch valve PVB was opened in the up and down directions of FIG. 1, and the pinch valves PV6, PV7 were opened.

Next, the ultrasonic processor 6 was driven, thus performing ultrasonic treatment of MLV (S240). Although ultrasonic treatment was carried out under appropriate conditions, for example, the output level was increased from 1 to 3, and driving for 1 min and stopping for 1 min were repeated 10 times.

Finally, SUV was recovered from the ultrasonic processor 6 (S250). In this step, while nitrogen gas was supplied into the ultrasonic processor 6, recovery was performed using the syringe pump SP6. Specifically, the port valves V4, V6 were opened (V3 was closed), the rotary valve RV2 was rotated to a position of 1-2 (the other rotary valves were in a state of the position 1 being closed), and the pinch valves PV6, PV7 were opened. While nitrogen gas was allowed to flow, the pinch valve PV5 was opened (PV4 was closed), and the rotary valve RV6 was rotated to a position of 3-4, so that the syringe pump SP6 was operated for suction purposes. Thereafter, the rotary valve RV6 was rotated to a position of 2-3, and thus the syringe pump SP6 was operated for discharge purposes, thereby recovering SUV into a bottle B6. Thereby, SUV having an average particle size of 53 nm could be manufactured.

2-2. Manufacturing of Water-Soluble Material-Enclosed SUV (Second Liposomes)

This manufacturing process was performed in the same manner as in the manufacturing process of 2-1, with the exception that the water-soluble material-enclosed MLV manufactured in 1-3 was used instead of the MLV used at S220. Thereby, water-soluble material-enclosed SUV could be manufactured.

As the water-soluble material, an enzyme (luciferase), an active ingredient (Barbital), an antigen (green fluorescent protein), an antibody (anti-green fluorescent protein antibody), or nucleic acid (pBR322 vector) was used, and the same operations as above were performed, thus manufacturing SUV in which the enzyme, active ingredient, antigen, antibody or nucleic acid was enclosed.

2-3. Manufacturing of SUV (Second Liposomes) having Oil-Soluble Material Enclosed in Membrane Thereof

This manufacturing process was performed in the same manner as in the manufacturing process of 2-1, with the exception that the MLV having oil-soluble material enclosed in the membrane thereof manufactured in 1-4 was used instead of the MLV used at S220. Thereby, SUV having oil-soluble material enclosed in the membrane thereof could be manufactured.

3-1. Manufacturing of Liposome Vaccine (Third Liposomes) Using Inactivated Virus

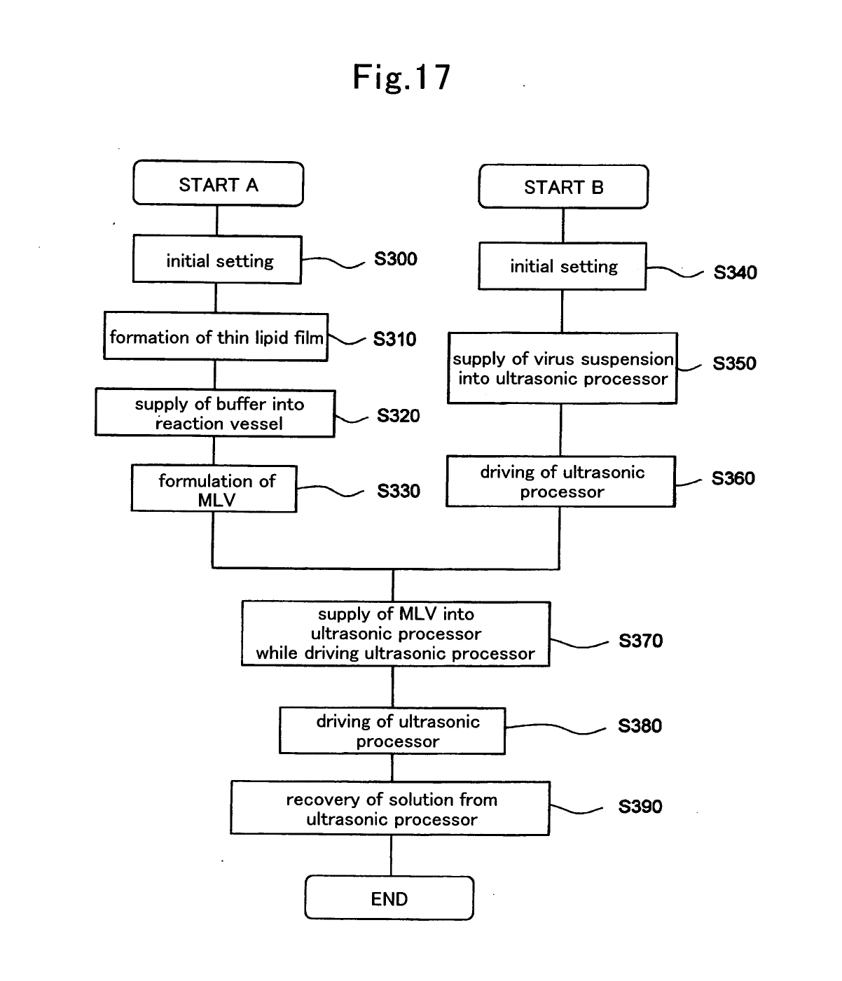

With reference to FIG. 17, a process for manufacturing liposome vaccine which is a kind of third liposome is described using inactivated Koi herpes virus. In this process, a virus suspension is subjected to ultrasonic treatment, and MLV may be added.

At the start A, initial setting (S300) was performed, after which a thin lipid film was formed using a reaction vessel 2 (S310). Next, a buffer was supplied into the reaction vessel 2 (S320), and an eccentric motor 3 was driven, thereby formulating MLV (S330). These steps (S300˜S330) were performed in the same manner as in S100˜S170 as above.

As a lipid chloroform solution, 2.5 ml of phospholipid (25 μmol dioleoylphosphatidyl choline and 2.5 μmol dioleoylphosphatidyl serine) dissolved in chloroform and 12.5 μmol cholesterol was used. Also as the buffer, 10.0 ml of 10 mM HEPES-NaOH/100 mM NaCl (pH 7.5) was used. Respective bottles were set, after which MLV was manufactured using the liquid crystal unit 15A of a computer 15.

On the other hand, at the start B, initial setting (S340) was performed, after which a virus suspension was supplied into an ultrasonic processor 6 (S350), and the ultrasonic processor 6 was driven (S360), thereby subjecting the virus to ultrasonic treatment. These steps (S340˜S360) were performed in the same manner as in S200˜S240 as above.

Next, while the ultrasonic processor 6 was driven, MLV was supplied into the ultrasonic processor 6 from the reaction vessel 2 (S370). This step was performed in the same manner as in S210˜S230 as above.

Next, predetermined ultrasonic treatment was performed, thereby formulating a Koi herpes virus liposome vaccine (S380). The liposome vaccine was observed using an optical microscope.

Finally, the liposome vaccine was recovered (S390). This step was performed in the same manner as in S250 as above. Thereby, the liposome vaccine was recovered into a bottle B6.

Typically, particles such as proteins diffused by ultrasonic treatment are re-agglutinated in a unit of some mm/sec. So, when particles which are easy to re-agglutinate in this way are used as an antigen, they should be mixed with liposomes immediately after ultrasonic treatment (or during ultrasonic treatment). A conventional device is difficult to adapt to such requirements, but the manufacturing device 1 according to the present embodiment can be used for the process shown in FIG. 17, whereby diffused particles are not re-agglutinated but are able to come into contact with liposomes.

3-2. Manufacturing of Liposome Vaccine (Third Liposome)

This manufacturing process was performed in the same manner as in the manufacturing process of 3-1, with the exception that a bacteria suspension was used instead of the virus suspension used at S350. The bacteria suspension was an E. coli suspension.

Consequently, liposome vaccine for bacteria could be manufactured.

3-3. Efficient Manufacturing of Liposomes (Third Liposomes)

Next, a process of efficiently manufacturing liposomes (in particular, SUV) is described. In this manufacturing process, S370˜S390 were performed after S300˜S330 of FIG. (i.e. start B˜S360 are omitted). Thereby, SUV can be efficiently manufactured.

Specifically, in the process of 3-1, S300˜S330 and S370˜S390 were performed (without start B S340˜S360).

Thereby, SUV could be efficiently manufactured.

3-4. Use as Bacteria Crusher

This manufacturing process was performed in the same manner as in the manufacturing process of 3-1, with the exception that a bacteria suspension was used instead of the virus suspension used at S350, and S300˜S330, S370, S380 were not performed. An E. coli suspension was used as the bacteria suspension.

Specifically, at the start B, S340, S350, S360, S390 were performed. Thereby bacteria could be destroyed using an ultrasonic processor.

In this way, the manufacturing device 1 according to the present embodiment could be used as a bacteria crusher.

4-1. Manufacturing of Peptide-Bound Liposomes (Fourth Liposomes) Using Eccentric Motor

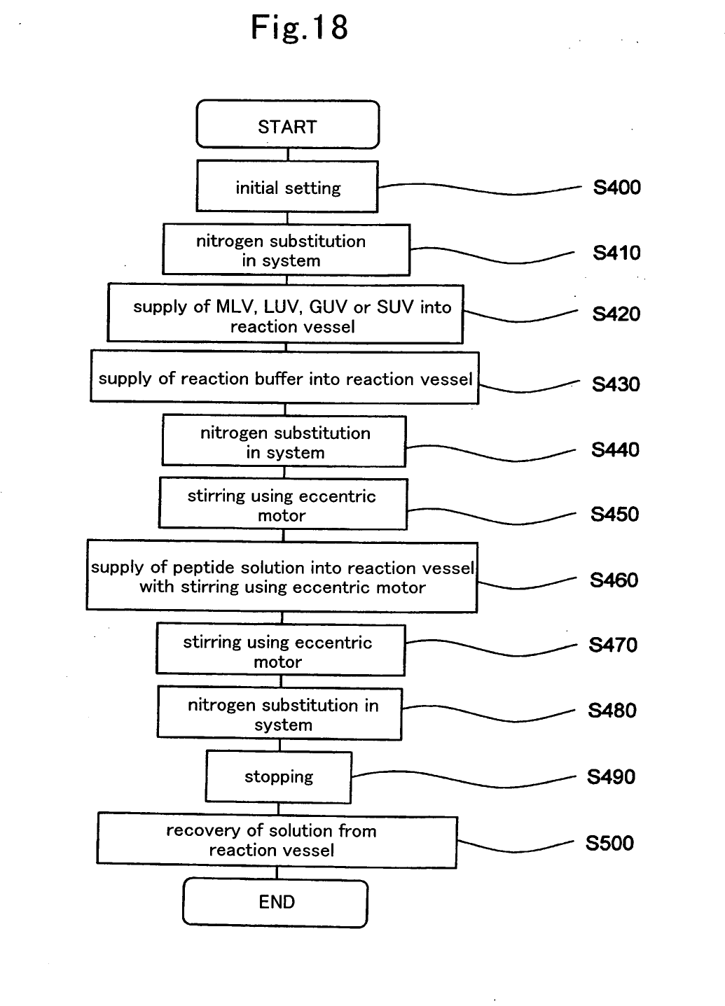

With reference to FIG. 18, a manufacturing process of peptide-bound liposomes which is a kind of fourth liposomes is described. This process was initiated using MLV, LUV, GUV or SUV recovered into an appropriate bottle after S160 or S240. The program set in the computer 15 was appropriately changed, whereby the present process was performed in a suitable initial state (e.g. MLV, LUV, GUV or SUV was previously added into the reaction vessel 2).

In this case, as the lipid chloroform solution, 2.5 ml of phospholipid (25 μmol dioleoylphosphatidyl choline, 25 μmol dioleoylphosphatidyl serine, 10 μmol NHS-distearoylphosphatidyl ethanolamine) dissolved in chloroform was used thus manufacturing a thin film, and MLV, LUV, GUV or SUV obtained from 10 ml of 10 mM acetic acid-Na acetate/175 mM NaCl (pH5.0) was used. NHS-DSPE reacts with an amino group of protein ‘peptide under a weakly alkaline condition (about pH 8.0) thus forming a covalent bond.

As the water-soluble peptide to be bound to the lipid bilayer membrane of liposomes, the peptide (Lys-Lys-ASP-Ser-Glu-Pro-Tyr:β-lipotropin segment) composed of seven amino acids described in SEQ ID No:1 was selected. This peptide was purchased from Sigma.

After the initial setting (S400), nitrogen substitution in a system was performed as in S110 (S410).

Next, MLV or SUV (liposomes), which were pre-formulated and then recovered in the bottle, were supplied into the reaction vessel 2 (S420). Next, 10 ml of a reaction buffer (100 mM Tris-HCl/100 mM NaCl (pH 8.0)) was supplied into the reaction vessel 2 (S430), and nitrogen substitution in a system was performed as in S110 (S440).

Next, the eccentric motor 3 was driven, and thus a vortex flow was generated in the liposomes inside the reaction vessel (S450). With stirring using the eccentric motor 3, the peptide solution was supplied into the reaction vessel 2 (S460), and the eccentric motor 3 was driven for a while so that liposomes were allowed to react with the peptide in the reaction vessel 2 (S470).

Next, nitrogen substitution in a system was performed as in S110 (S480), and operation of the eccentric motor 3 was stopped (S490).

Finally, the solution in the reaction vessel 2 was recovered (S500).

Thereby, peptide-bound liposomes (fourth liposomes) having a bound ratio of about 50% could be manufactured.

4-2. Manufacturing of Protein (Antigen)-Bound Liposomes (Fourth Liposomes)

This manufacturing process was performed in the same manner as in the manufacturing process of 4-1, with the exception that a protein (antigen) solution was used instead of the peptide solution used at S460. As the protein (antigen) solution, green fluorescent protein dissolved in a buffer was used. Consequently, protein (antigen)-bound liposomes could be manufactured.

4-3. Manufacturing of Nucleic Acid-Bound Liposomes (Fourth Liposomes)

This manufacturing process was performed in the same manner as in the manufacturing process of 4-1, with the exception that cationic liposomes were used instead of MLV, :UV, GUV or SUV used at S420, and a nucleic acid solution was used instead of the peptide solution used at S460. As the cationic liposomes, lipofectamine (available from Invitrogen) was used, and as the nucleic acid solution was used a pBR322 vector dissolved in a buffer.

Consequently, nucleic acid-bound liposomes could be manufactured.

4-4. Manufacturing of Recombinant Proteoliposomes (Fourth Liposomes)

This manufacturing process was performed in the same manner as in the manufacturing process of 4-1, with the exception that a reaction buffer (10 mM CH₃COOH—CH₃COONa/10 mM NaCl (pH 4.0)) was used instead of the reaction buffer (100 mM Tris-HCl/1.00 mM NaCl (pH 8.0)) used at S430, and a membrane protein-loaded baculovirus suspension was used instead of the peptide solution used at S460.

The membrane protein-loaded baculovirus suspension was one manufactured by a technique disclosed in a Patent Application of the present inventors (WO2007/094395-A1).

Consequently, recombinant proteoliposomes could be manufactured.

4-4. Use as Bioreactor

An example of using the manufacturing device 1 according to the present embodiment as a bioreactor is described.

This manufacturing process was performed in the same manner as in the manufacturing process of 4-1, with the exception that a reaction buffer (10 mM CH₃COOH—CH₃COONa/10 mM NaCl (pH 5.6)) was used instead of the reaction buffer (100 mM Tris-HCl/100 mM NaCl (pH 8.0)) used at S430 and a phospholipase D (sigma P8804) solution was used instead of the peptide solution used at S460. In the present process, MLV, LUV, GUV or SUV of S420 was replaced with LUV or SUV.

By the present process, only the outer compartment of the lipid bilayer membrane was converted from PC (phosphatidylcholine) into PA (phosphatidic acid).

In this way, the manufacturing device 1 according to the present embodiment could be used as a bioreactor.

According to the present embodiment, the manufacturing device 1 for rapidly and efficiently creating large amounts of many kinds of liposomes can be provided. The manufacturing device 1 includes a combination of a relatively small mechanical unit comprising the cylindrical reaction vessel 2 and the eccentric motor 3 and an ultrasonic processor 6 and is automatically controlled by the computer 15, thereby rapidly and efficiently manufacturing a variety of liposomes (in particular, liposome vaccine).

### DESCRIPTION OF REFERENCE NUMERALS

1—automatic multifunctional liposome manufacturing device (automatic liposome vaccine manufacturing device)

2—reaction vessel

2A—reaction space

3—eccentric motor

4—heater

5—temperature sensor

6—ultrasonic processor

9—inert gas bomb

10—vacuum pump

11—organic solvent recovery unit

15—computer

24—shaking maintenance unit

B1˜B10—bottle

SP1˜SP7—syringe pump

T41—line (decompression line)

T42—line (inert gas line)

T43—line (inert gas line)

1. An automatic multifunctional liposome manufacturing device, comprising:
a cylindrical reaction vessel;
an eccentric motor for generating a vortex flow in a solution stored in a reaction space of the reaction vessel;
a heater for heating the reaction vessel to a predetermined temperature;
an aqueous solution line provided to the reaction vessel so as to introduce an aqueous solution into the reaction space;
a first bottle provided on an end of the aqueous solution line so as to store the aqueous solution;
a first pump for transferring the aqueous solution into the reaction space via the aqueous solution line from the first bottle;
an inert gas line provided to the reaction vessel so as to introduce an inert gas into the reaction space;
a decompression line for decompressing the reaction space;
a vacuum pump for decompressing the reaction space via the decompression line;
a lipid line provided to the reaction vessel so as to introduce an organic solvent having lipid dissolved therein into the reaction space;
a second bottle provided on an end of the lipid line so as to store the organic solvent;
a second pump for transferring the organic solvent into the reaction space via the lipid line from the second bottle;
an organic solvent recovery unit for recovering the organic solvent; and
a computer for controlling the eccentric motor, the heater, the first pump and the second pump,
wherein under control of the computer, the inert gas is introduced into the reaction vessel, and the eccentric motor is driven, so that a vortex flow is generated in the organic solvent having lipid dissolved therein that was put in the reaction space, and the reaction space is decompressed and thus the organic solvent is gasified in the reaction space and recovered by the organic solvent recovery unit, thus forming a thin lipid film on the inner wall of the reaction vessel, and then the inert gas is introduced into the reaction space, and the aqueous solution is introduced into the reaction vessel, the eccentric motor is driven to thus generate a vortex flow in the aqueous solution, thereby manufacturing liposomes from the thin lipid film and the aqueous solution.

2. The automatic multifunctional liposome manufacturing device of claim 1,
wherein the aqueous solution line branches into a plurality of lines at an end opposite the reaction vessel, and an end of each of the plurality of lines is equipped with a water-based bottle for storing a solvent composed mainly of water and a water-based pump for transferring the solvent into the reaction space via the aqueous solution line from the water-based bottle,
and each water-based pump is controllable by the computer,
and the lipid line branches into a plurality of lines at an end opposite the reaction vessel, and an end of each of the plurality of lines is equipped with an organic-based bottle for storing a solvent composed mainly of an organic solvent and an organic-based pump for transferring the solvent into the reaction space via the lipid line from the organic-based bottle,
and each organic-based pump is controllable by the computer.

3. The automatic multifunctional liposome manufacturing device of claim 1,
wherein a solution transfer line for sucking the aqueous solution stored in the reaction so as to transfer it, and a transfer pump controlled by the computer so as to suck the aqueous solution are provided,
and an end of the solution transfer line is equipped with an ultrasonic processor controlled by the computer so as to irradiate the aqueous solution with ultrasonic waves.

4. A method of manufacturing liposomes using the automatic multifunctional liposome manufacturing device of claim 1, comprising:
(1) introducing an inert gas into a reaction space of a reaction vessel, and inside the reaction space, while generating a vortex flow in an organic sol vent having lipid dissolved therein that was put in the reaction space, decompressing the reaction space, so that the organic solvent is gasified in the reaction space, thus forming a thin lipid film on the inner wall of the reaction vessel; and
(2) introducing the inert gas into the reaction space, adding an aqueous liquid to the thin lipid film, and generating a vortex flow in the aqueous liquid inside the reaction space, thereby formulating first liposomes.

5. A method of manufacturing liposomes using the automatic multifunctional liposome manufacturing device of claim 3, comprising:
(1) introducing an inert gas into a reaction space of a reaction vessel, and inside the reaction space, while generating a vortex flow in an organic solvent having lipid dissolved therein that was put in the reaction space, decompressing the reaction space, so that the organic solvent is gasified in the reaction space, thus forming a thin lipid film on the inner wall of the reaction vessel;
(2) introducing the inert gas into the reaction space, adding an aqueous liquid to the thin lipid film, and generating a vortex flow in the aqueous liquid inside the reaction space, thus formulating first liposomes; and
(3) irradiating the first liposomes with ultrasonic waves using the ultrasonic processor, thereby formulating second liposomes.

6. A method of manufacturing liposomes using the automatic multifunctional liposome manufacturing device of claim 3, comprising:
(1) introducing an inert gas into a reaction space of a reaction vessel, and inside the reaction space, while generating a vortex flow in an organic solvent having lipid dissolved therein which was put in the reaction space, decompressing the reaction space, so that the organic solvent is gasified in the reaction space, thus forming a thin lipid film on the inner wall of the reaction vessel;
(2) introducing the inert gas into the reaction space, adding an aqueous liquid to the thin lipid film, and generating a vortex flow in the aqueous liquid inside the reaction space, thus formulating first liposomes; and
(4) irradiating a suspension liquid with ultrasonic waves using the ultrasonic processor and adding the first liposomes to the ultrasonic processor, thereby formulating third liposomes.

7. A method of manufacturing fourth liposomes from the liposomes manufactured according to any one of claims 4 to 6 comprising:
(5) introducing an inert gas into a reaction space, and while generating a vortex flow in a suspension of any one kind of liposomes among first liposomes˜third liposomes, adding an aqueous liquid, thereby formulating the fourth liposomes inside the reaction space.
